(19) United States
(12) Patent Application Publication
(10) Pub. No.: US 2020/0100934 A1
(43) Pub. Date: Apr. 2, 2020

(54) SKIN COOLING APPARATUS AND METHODS

(71) Applicants: Anthony Ariano, Richmond Hill, GA (US); Ben Spitalnick, Savannah, GA (US); Keith Matthew Seibert, Savannah, GA (US)

(72) Inventors: Anthony Ariano, Richmond Hill, GA (US); Ben Spitalnick, Savannah, GA (US); Keith Matthew Seibert, Savannah, GA (US)

(21) Appl. No.: 16/146,089

(22) Filed: Sep. 28, 2018

(51) Int. Cl. A61F 7/02; A61M 5/42; B05B 7/06; A61M 19/00

(52) U.S. Cl. CPC A61F 7/02; A61M 5/422; A61F 2007/0001; A61M 19/00; B05B 7/061

(57) ABSTRACT

A lightweight, portable, cost-effective skin-cooling device is adapted for use with a coolant spray in an existing canister having a rim surrounding a push-activated spray tube. The apparatus comprises a housing with an inlet end that snaps onto the existing canister, an outlet end with a skin-contacting component, and a pushbutton. A tubular structure, disposed within the housing, includes an inlet end configured to engage with the push-activated spray tube of the canister, and an outlet end directed toward the skin-contacting component supported over the outlet end of the housing. Depressing the pushbutton causes the inlet end of the tubular structure to depress the push-activated spray tube, thereby causing the coolant spray to be carried though the tubular and out the outlet end thereof to cool the skin-contacting component.

### FIELD OF THE INVENTION

This invention relates generally to skin and subcutaneous tissue cooling and treatment and, in particular, to devices and methods that use cold temperatures for needle injections, cosmetic uses and localized pain relief.

### BACKGROUND OF THE INVENTION

There are many reasons to reduce skin and tissue temperatures, and there are many approaches and procedures used to achieve this goal. Injections of anesthetics such as lidocaine can dramatically reduce pain associated with skin procedures, but the discomfort of the injection can be as painful as the procedure itself. As such, there remains an outstanding desire to numb the skin and tissues without inflicting pain in the process.

With specific regard to needle injections, alternatives include needle-free syringes that deliver vaccines through a quick, fluid stream that penetrates the skin, and medicated sprays that are puffed into the nostrils. There are also hand-held devices that track veins and the direction of blood flow on an imaging device, giving users a real-time digital image of the patient's blood pattern to track especially difficult veins needed for medical procedures and injections.

Topical anesthetics have been widely researched, but they have proven to be largely ineffective in many situations. Although topical anesthetics can take the sting out of needle punctures, they have not been routinely used for immunizations or blood draws due to high cost and they inconvenience associated with waiting for them to take effect.

Vapocoolants and anesthetic gels containing amethocaine are cheaper and work more quickly than topical anesthesics. Vapocoolants halt pain signals through rapid cooling. These sprays contain chemicals that evaporate quickly under normal room conditions. When applied topically, they extract heat from the skin as they evaporate. Vapocoolants are inexpensive and provide nearly instant skin cooling. However, since numbness lasts only a few seconds, they are appropriate only for procedures that can be completed quickly. Some patients may find vapocoolants uncomfortable, and the coolants must be applied with care to avoid freezing the skin, which could cause blistering or even frostbite. There may be environmental drawbacks if the vapocoolant releases chlorofluorocarbons, and some vapocoolants are flammable.

Skin cooling is also known to reduce the volume of subcutaneous fat, thereby achieving pain relief. In this regard, one alternative includes immersive cooling. However, the equipment associated with such techniques can be costly, and the procedures present some risks of cold injury.

### SUMMARY OF THE INVENTION

This invention addresses and solves problems associated with prior-art skin skin/tissue cooling apparatus by providing an extremely cost-effective article adapted for use with a coolant spray in an existing canister. The apparatus comprises a housing with an inlet end and an outlet end. The inlet end of the housing includes a structure enabling the housing to be removably coupled (i.e, snapped on) to the rim of the existing canister. A component supported over the outlet end of the housing is adapted for skin contact. When a user presses a button on the housing, the coolant spray is directed within the housing to the component supported on the outlet of the housing, causing the component to become cool, which is placed against a region of skin to be cooled.

A tubular structure, disposed within the housing, includes an inlet end configured to engage with the push-activated spray tube of the canister, and an outlet end directed toward the skin-contacting component supported over the outlet end of the housing. A pushbutton, accessible on the outer surface of the housing, is coupled to the tubular structure, such that depressing the pushbutton causes the inlet end of the tubular structure to depress the push-activated spray tube, thereby causing the coolant spray to be carried though the tubular structure and out the outlet end thereof to cool the skin-contacting component.

In a preferred embodiment, the canister is a conventional compressed-air canister having a 1-inch or other diameter circular rolled rim or ‘doughnut.’ The inlet end of the housing is likewise circular, and configured to removably coupled to the canister by way of a series of barbs or projections that engages with the circular rim of the canister. The outlet end of the housing may be at an angle relative to the inlet end such a ninety degrees, with the pushbutton being conveniently accessible on an upper surface of the housing.

The tubular structure may be coupled to the housing by way of a living hinge, such that depressing the pushbutton causes the entire tubular structure to move. This also enables the entire housing, including the pushbutton structure, to be made with as few as a single injection-molded plastic piece. The skin-contacting component is preferably a round disk of aluminum or other thermally conductive material.

### BRIEF DESCRIPTION OF THE DRAWINGS

FIG. 1 is a perspective view of a preferred embodiment of the invention coupled to an existing spray canister;

FIG. 2 is a cross section of the preferred embodiment depicted in FIG. 1;

FIG. 3A is a cross section of the invention without a canister of coolant spray and without a cooling disk component prior to activation of the pushbutton;

FIG. 3B is a cross section of the invention without a canister of coolant spray and without a cooling disk component following activation of the pushbutton;

FIG. 4A is a front view of a disc-shaped cooling component;

FIG. 4B is a cross section of the disc-shaped cooling component of FIG. 4A;

FIG. 4C is a back view of the disc-shaped cooling component of FIG. 4A; and

FIG. 5 is a perspective view of a cosmetic embodiment of the invention intended for subcutaneous tissue cooling.

### DETAILED DESCRIPTION OF THE PREFERRED EMBODIMENTS

This invention resides in an article that couples to an existing canister of coolant spray to cool skin surfaces to reduce or eliminate pain. While geared towards children and adults with needle phobia, it is applicable to a wide range of users including pediatricians to diabetics who inject themselves on a daily basis. Another application is for cooling the skin instantly after a first or second degree burn or sprain as opposed to, or in conjunction with, applying an ice pack. It may further be used as a cosmetic device to cool the skin and tissues in the facial area, for example, to low temperatures to help the skin maintain a youthful appearance.

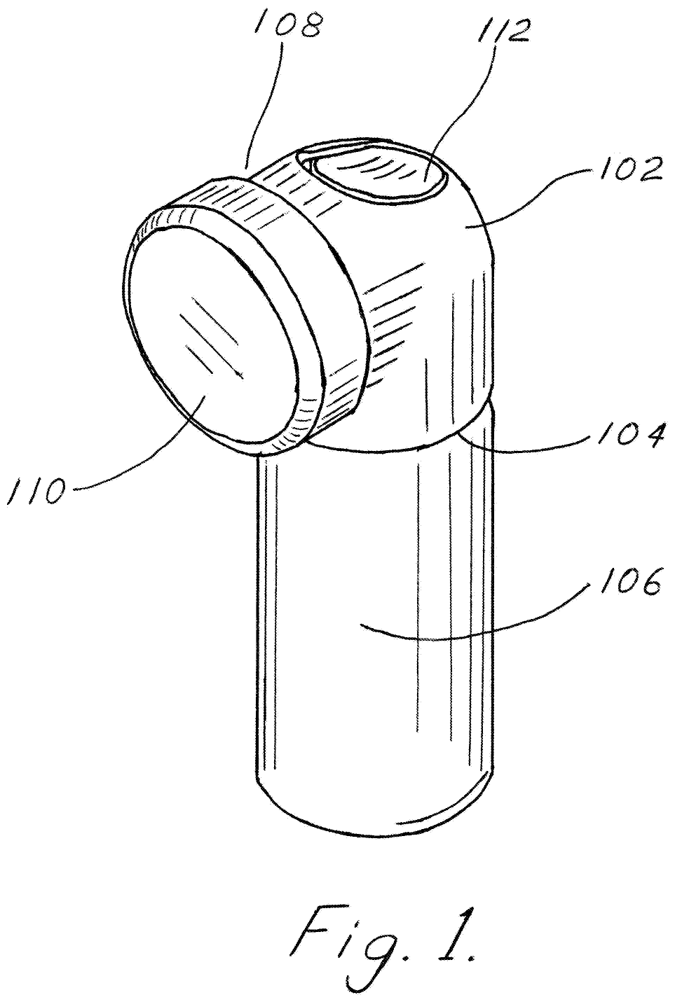

FIG. 1 is a simplified perspective view of a preferred embodiment of the invention. The article comprises a generally hollow housing 102 having an inlet end 104 coupled an existing canister of coolant spray 106, and an outlet end 108 that receives a skin-contacting component 110. As described in further detail below, when a pushbutton 112 on the housing is depressed, coolant spray from canister 106 is directed internally to the housing toward the component 110, cooling the component down for skin contact.

In the preferred embodiment, the housing 102 is molded from a single piece of injection-molded plastic, and the skin-contacting component 110 is a thermally conductive disc. The disk may be round, and may be constructed from a metal such as aluminum. The invention is not limited in these aspects, however, as multiple pieces may be used to form the housing, and the disc 110 made be made in other shapes and from other materials, including layered composites.

Different canisters with different coolant sprays may also be used, though in the preferred embodiment, a canister of “compressed air” is used due to its inertness. While compressed air is not generally known as a coolant, when the canister is tilted, the propellant forms a rapidly cooling spray that the invention uses to advantage. In the most preferred embodiments, the invention uses a standard or conventional canister having a circular, 1-inch diameter rolled rim or “doughnut” ordinarily used to retain the cap of the canister. In accordance with the method of use, the plunger cap is removed, exposing a push-activated spray tube 202 extending from the canister.

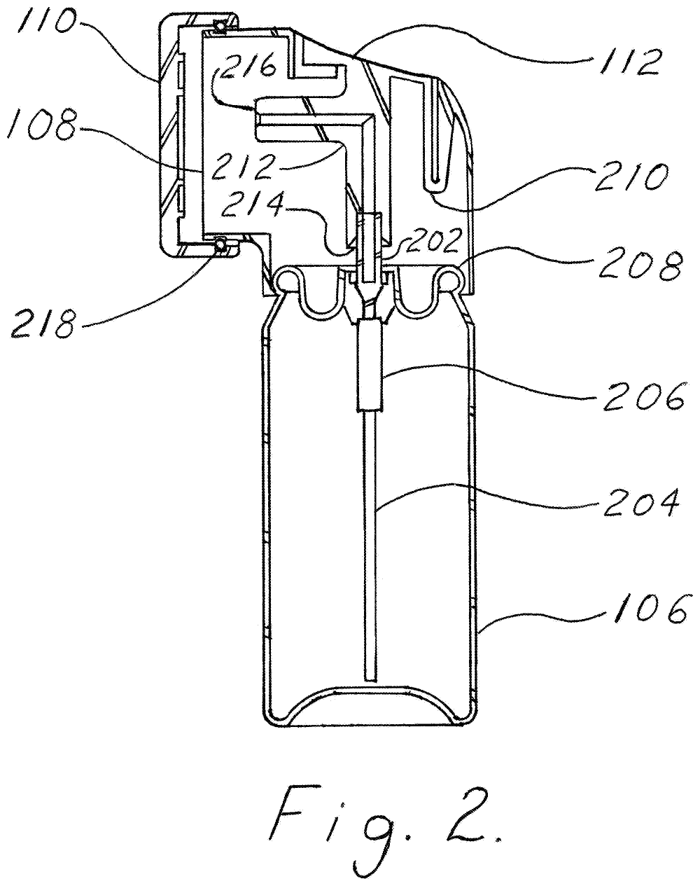

FIG. 2 is a cross section of the embodiment depicted generally at 102, coupled to an existing canister 106 and including a skin-contacting component 110. Component 110, a circular disc, may be attached to the outlet end of housing by way of an O-ring 218 that fits between opposing circumferential grooves on the outer surface of the outlet end 218 and the inner surface of the component 110. Other attachment mechanisms, such as bayonet, screw-on, etc., may alternatively be used.

In FIG. 2, the cap and spray button of the canister have been removed, exposing a tube 202 in communication with a dip tube 204 through a spray mechanism 206. The canister 106 comes with a rolled rim 208, to which the article 102 removably attaches. Regardless of the spray mechanism provided, when the tube 202 is depressed, coolant from the canister 106 flows up through the dip tube 204 and into a tubular structure 212 disposed within the article housing 102.

The tubular structure 212 includes an inlet end 214 configured to engage with the push-activated spray tube 218 of the canister, and an outlet end 216 directed toward the skin-contacting component 110 supported over the outlet end 108 of the housing 102. The pushbutton 112 is coupled to the tubular structure 212 in a manner whereby depressing the pushbutton causes the inlet end of the tubular structure to depress the push-activated spray tube 202, thereby causing the coolant to be carried though the tubular structure 212, and out the outlet end 216 thereof, to cool the skin-contacting component 110.

In the preferred embodiment, the outlet end 108 of the housing at an angle relative to the inlet end such as a right angle. Such a construction enables the pushbutton 112 to be conveniently located on the top of the housing, such that when a user grasps the canister with their hand, the index finger may easily be used to depress the button 112. Further in accordance with the preferred embodiment, the tubular structure 212 is coupled to the housing by way of a living hinge 210, such that depressing the pushbutton 112 causes the entire tubular structure 212 to move.

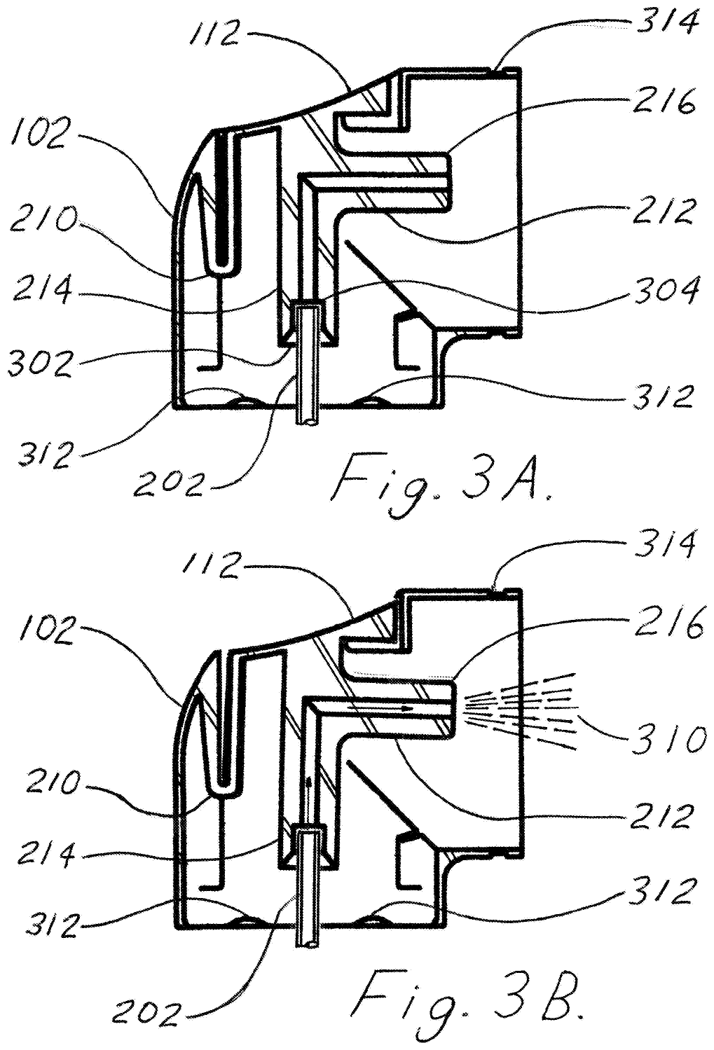

FIG. 3A is a cross section of the invention without attachment to a canister of coolant spray, and without a cooling disk component prior to activation of the pushbutton. FIG. 3B is a cross section illustrating activation of the pushbutton 112. The inlet end 214 of tubular structure 212 preferably includes a cavity 304 that receives the spray tube 202 of the existing canister. To assist with alignment during attachment of the housing 102, the inlet end 214 may include a flared opening 302.

As shown in FIG. 3B, depression of button 112 causes living hinge 210 to slightly separate, further causing the inlet end 214 of tubular structure 212 to move downwardly. This, in turn, causes spray tube to activate the spray mechanism of the canister, releasing coolant spray 310 out the outlet end 216 of tubular structure 212 toward the cooling disc component (not shown in FIGS. 3A, B), FIGS. 3A, B show some of inwardly directed barbs or protrusions 312 that engage with the rim 208 of the canister, enabling the housing to firmly snap onto the top of the canister. FIGS. 3A, B also show the outer, circumferential groove 314 that receives O-ring 218 for attachment of the cooling disc component 110 to the outlet end of housing 102.

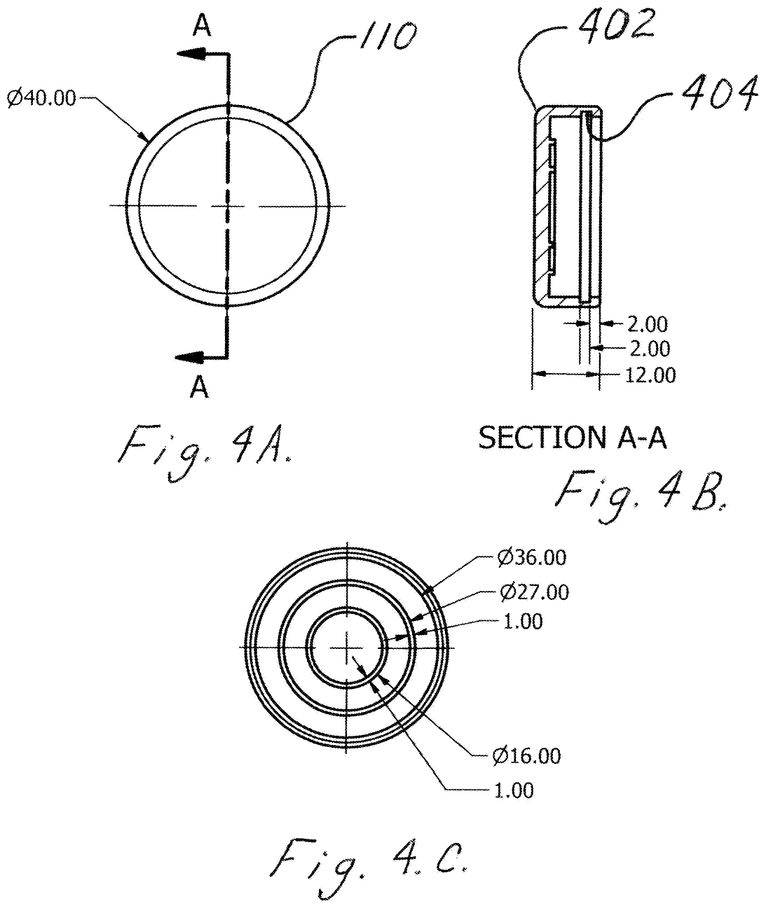

FIGS. 4A-C are drawings that illustrate a preferred disc-shaped cooling component 110. FIG. 4A is a front view, FIG. 4B is a cross sectional view, and FIG. 4C is a back view. The dimensions, in millimeters, are nominal, and represent one possible structure, without limitation. Again, this component may be constructed from aluminum or other suitable thermally conductive material(s). The front preferably has a rounded edge 402 for smooth skin contact, and the back may include an irregular surface to improve the distribution of the coolant spray. In particular, the backside of the disc 110 may include circular grooves or rings, as shown. FIG. 4B shows an inner circumferential groove 404 that receives O-ring 218 for attachment of the cooling disc component 110 to the outlet end of housing 102.

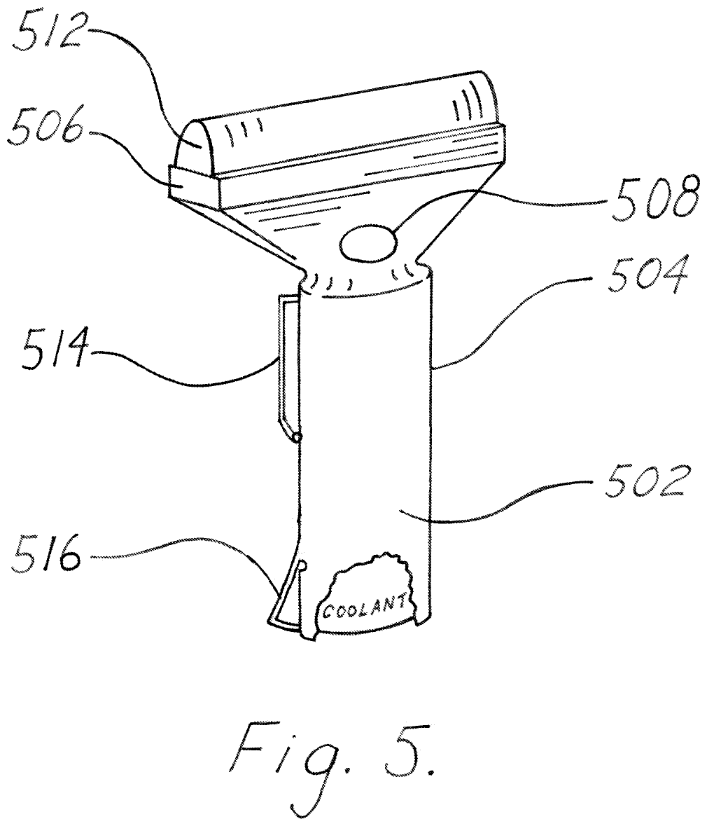

While the preferred embodiment includes a cooling disc, other embodiments possible in accordance with the invention for other purposes. For example, FIG. 5 is a perspective view of a cosmetic embodiment 502 of the invention intended for skin, subcutaneous and deep-tissue cooling. In contrast to an angled housing and circular disc, the attachment 502 includes an elongated handle 504 that flares at 506 to a rectangular cavity that receives an elongated cooling component in the form of an applicator 512 with a rounded edge. In use, the cooled edge is moved along the skin to create cosmetic improvement.

In all embodiments of the invention, including the cosmetic embodiment, the skin-contacting component is typically cooled in advance of application to prepare the device for use. Once the cooled component is in contact with the skin, however, the additional coolant may be administered to re-cool the component or maintain the component at a desired temperature. The mechanism to release the cooling spray may be in the form of an electromechanical push-button mechanism 508. It may also be in the form of a mechanical lever release 514 or living hinge squeeze mechanism 516. Any of these can function by causing repetitive intermittent release of coolant. In some embodiments, a liquid-crystal or other material may be used in conjunction with the cooled, skin-contacting component to give a user an indication of application temperature.

1. Skin cooling apparatus adapted for use with a coolant spray in an existing canister having a rim surrounding a push-activated spray tube, the apparatus comprising:
a housing having an outer surface, an inlet end and an outlet end;
a skin-contacting component supported over the outlet end of the housing;
wherein the inlet end of the housing includes a structure enabling the inlet end to be removably coupled to the rim of the existing canister;
a tubular structure disposed within the housing, the tubular structure having an inlet end configured to engage with the push-activated spray tube of the canister, and an outlet end directed toward the skin-contacting component supported over the outlet end of the housing; and
a pushbutton accessible on the outer surface of the housing, the pushbutton being coupled to the tubular structure disposed within the housing, such that depressing the pushbutton causes the inlet end of the tubular structure to depress the push-activated spray tube, thereby causing the coolant spray to be carried though the tubular and out the outlet end thereof to cool the skin-contacting component.

2. The apparatus of claim 1, wherein the inlet end of the housing is circular and configured to removably couple to a circular rim of the existing canister.

3. The apparatus of claim 1, wherein the inlet end of the housing is circular and configured to removably couple to an existing canister having a one-inch diameter circular rim.

4. The apparatus of claim 1, wherein the inlet end of the housing is configured to removably couple to an existing compressed-air canister.

5. The apparatus of claim 1, wherein:
the outlet end of the housing is circular; and
the skin-contacting component is a circular disc made from aluminum or other thermally conductive material.

6. The apparatus of claim 1, wherein the outlet end of the housing is at a non-zero angle relative to the inlet end.

7. The apparatus of claim 1, wherein the outlet end of the housing is at a ninety degree angle relative to the inlet end.

8. The apparatus of claim 1, wherein the pushbutton is on an outer surface of the housing.

9. The apparatus of claim 1, wherein the tubular structure is coupled to the housing by way of a living hinge, such that depressing the pushbutton causes the entire tubular structure to move.

10. The apparatus of claim 1, wherein the skin-contacting component is an elongated component with a rounded edge for movement against the skin for cosmetic purposes.
